(19) United States
(12) Patent Application Publication
(10) Pub. No.: US 2004/0256329 A1
(43) Pub. Date: Dec. 23, 2004

(54) Photodynamic biological fluid pathogen inactivation/filtering apparatus and method

(76) Inventors: Peter Meserol, Mine Hill, NJ (US); Jesse Acker, Hackettstown, NJ (US); Rita Prodell, West Orange, NJ (US); Lawrence Lenart, Clifton, NJ (US); Robert Schenck, East Rutherford, NJ (US); Shirley Meserol, Mine Hill, NJ (US)

Correspondence Address: William Squire Esq., c/o Carella, Byrne, Bain, Gilfillan,, Cecchi, Stewart & Olstein, 5 Becker Farm Road, Roseland, NJ, 07068, US

(21) Appl. No.: 10/863,163

(22) Filed: Jun. 8, 2004

Related U.S. Application Data
(60) Provisional application No. 60/478,443, filed on Jun. 13, 2003.

(51) Int. Cl. C02F 1/30

(52) U.S. Cl. 210/748; 210/205

(57) ABSTRACT

A tube has a plurality of sections, several with a transparent wall and a static mixer in each section, which mixers may also be transparent. The initial section mixes a photo inactivation agent with a body fluid such as blood and so on. The static mixers each have adjacent static mixing elements including a pair of axially aligned static mixing sections each formed of a 180° helix, adjacent sections being relatively rotated 90°, all of the elements being aligned in a corresponding tube section and having the same orientation. The apparatus is disposable and includes fluid supply containers, tubing, valves, sensors, and mixing devices coupled to pumps, all under control of a CPU. A static mixer filter filters the processed fluid and may contain porous resin particles coated on the inner surfaces of or packed into a mixing device, and/or coated on a porous membrane in a mixing device. Light is supplied by high power LEDs which may include cooling devices and reflectors for photo inactivation of the agent.

This application claims the benefit of provisional application Ser. No. 60/478,443 filed Jun. 13, 2003, entitled “Photodynamic Biological Fluid Pathogen Inactivation/Filtering Apparatus and Method” incorporated by reference herein in its entirety.

This invention relates to an apparatus and method for the inactivation of infectious agents and/or pathogens/contamination in biological fluids and filtering to remove contaminants from the treated biological fluid.

### BACKGROUND OF THE INVENTION

Attention in the recent years has been placed on the testing of the human blood supply for many of the infectious agents found in patient samples to assure that transfused blood is as free of pathogens as the current state of technology detection allows. Such pathogens as related to AIDS and hepatitis, for example, are the cause of major epidemics. The rejection of blood units containing these pathogens seriously depletes the available blood for transfusion. Furthermore, tests for infectious agents are not 100% accurate in detection of infectious agents. The existence of these tests has dramatically reduced blood borne infections due to transfusions, but additional precautions are needed to increase both confidence in the available blood supply as well as to reduce the incidence of infection and save lives.

Photodynamic therapy has a long history in the medical field. In the 1980's Dr. Thomas J. Dougherty of the Roswell Park Cancer Institute experimented with porphyrin derivatives to examine their effects on cancer cells and solid tumors. Much work has been done in this field following Dr. Dougherty's publication of results. Initially, emphasis was placed on the destruction of solid tumors where they could be reached by fiber optic coupled lasers. Porphyrins have been found particularly useful in that they preferentially gravitate to rapidly dividing cells. Thus they tend to congregate in tumors.

In collaboration with Dr. Dougherty, Johnson and Johnson (J&J) developed a system for using photodynamic principles to cleanse plasma of bacteria and viruses. This development has been followed up by a number of patents relating to the photo-inactivation of viruses and bacteria in blood and blood products.

The basic procedure followed by many of the prior art approaches involves the addition of a photo-inactivation agent to a biological sample and then exposing the sample to an intense light to activate the agent.

An early approach was to premix the photo-agent in a relatively transparent treatment bag. The treatment bag is then placed in a high intensity light field containing sufficient energy to activate the photo agent. It would also be necessary to agitate the treatment bag in order to assure that all of the sample had absorbed adequate radiation quanta over the time of the treatment.

This technique, while sometimes effective after a long period of agitation (the time factor being subjective) often produces erratic results due to the poor mixing of the components of the mixture and the non-reproducibility of the light exposure onto the samples.

Bag-based dispersion kinetics as in common use, and many concurrent photo-illumination schemes, are of questionable consistency, efficiency, and most importantly, reproducibility. Fluid mixing in intravenous (I.V.) bags has been studied in the pharmacy literature; the performance of flexible walled containers as vehicles for admixture dispersion is broadly addressed in publications from the paint and adhesive industry.

The literature describes that there is inadequate mixing of aqueous (non-colloidal) pharmaceuticals in I.V. bags and reinforce this result. Little is known about blood/agent admixtures.

Rocking, tumbling and/or squeezing flexible containers is inefficient when mixtures of particles (such as red blood cells (RBC's)) in fluids are to be evenly dispersed. Efficient mixing dynamics are functions of fixed container geometry whereas a flexible bag is a variable geometry. Full mixing time is highly variable, and with colloidal dispersions may be protracted. Adequate and thorough admixture is not easily validated.

Even though the biological sample contains an inactivating agent of the proper concentration, the inconsistency of the dispersion of the involved fluids illuminated by the light source coupled with the random reflections of the light source off the randomly moving surface of the containment bag produces inconsistent results. In a bag type system, as the bag flexes, the outer surface presents a curved, moving and random surface which reflects a large portion of light flux from the inactivation light source. Applying an agent of appropriate values and the inconsistency of dispersion of the involved fluids is the concurrent variable as well as the random exposure of the cellular admixture to activating light. In a bag type system, as the bag flexes, the outer surface presents a curved, moving, and random surface which reflects a large portion of light flux from an inactivation light source spaced from the bag. Only a fraction of the light “couples” to the bag and is transmitted to the contents for substantive inactivation of pathogens in the contents. Illumination and dispersion are not linked geometrically and the photo-activation of the chemical, as well as its distribution, is essentially chaotic.

The above described bag method of mixing has a second disadvantage. The requirement of a drive motor and large moving components is a substantial cost, safety, and service liability to be advantageously avoided. A “brute force” illumination scheme, i.e., excess illumination than ordinarily needed for the task, is required to compensate for the optical power losses in both the variable “target” geometry and the separation required between the light sources (a fixed geometry) and moving bag. The commercial reliability of such a process is believed questionable.

The prior art in photo inactivation of blood deal primarily with the proprietary agents used and most describe systems in which the proprietary agent is mixed with blood in a container and then illuminated by various light sources to activate the agent. During this process, the container, or blood bag is continually agitated to attempt to effect uniform illumination of the contents of the bag over the period of treatment. There are several flow/illumination schemes disclosed, but all suffer from insufficient mixing during illumination. Higher illumination intensity light sources, than otherwise needed, are employed to compensate for the poor mixing. These light sources have the disadvantage of excessive heating of the sample, which is not desirable.

Wolf et al., U.S. Pat. No. 5,290,221, describes a flow through system using a cooled linear light source located remote from the treatment bag. The light, an incandescent filament (a notorious source of excessive heat), is placed remote because of excess heating and various reflective schemes are employed to concentrate the light onto the treatment bag. The treatment bag is similar to a commercial blood bank heating bag having an arcuate path for the fluid flow as it is exposed to the incident radiation. While more efficient in exposing a thin layer of fluid to the light, this approach suffers from inadequate mixing and the interchange of layers of the involved fluid.

The result is that not all of the sample receives the same radiation. To compensate for uneven exposure and highly inefficient optical coupling between the light source and the treatment bag, very high intensity light sources are required with their concomitant undesirable high heat output. This system requires appreciable heat removal to maintain the blood within safe temperature limits as well as to increase the life of the light source.

Goodrich et al., U.S. Pat. No. 6,277,337, discloses primarily the use of endogenous photoinactivators as the material of concern and does not deal with apparatus in any meaningful fashion other than as a sketch of how the photoinactivation might be implemented. The primary advantage of this method is that the material is derived from natural chemicals found in the body and ostensibly one does not have to remove this substance after photo-inactivation processing.

Hearst et al., U.S. Pat. No. 5,854,967, defines a system with a series of discrete samples in tubes immersed in cooling water to prevent irradiation heating from the light source.

Chapman et al., U.S. Pat. No. 5,866,074, describes a system using a whole bag irradiation system in which measurements are made of absorbance pre and post irradiation. The measurements define the extent to which the sample has been effectively irradiated. This system suffers from the deficiencies described herein, i.e., inadequate mixing in a treatment bag.

Other photo inactivation apparatus approaches involve such carriers as labyrinthine bags where blood flows through a tortuous path in a thin sheet exposed to light in an attempt to illuminate as much as possible of the sample. This approach also suffers from non-reproducible flow and mixing.

### SUMMARY OF THE INVENTION

The present inventors have discovered, as discussed above, that the problem of the poor treatment of fluid in the prior art is the inadequate mixing of the involved fluid. The apparatus of the present invention solves this problem by providing a mixing of the fluid that is more uniform and thorough than the prior art body fluid mixing devices. This apparatus thus provides more thorough and uniform photo-illumination of the fluid. Such more thorough uniform photo-illumination ensures the incident light is substantially more uniformly applied to the fluid throughout the fluid than otherwise provided by the prior art. Thus reliable and more thorough inactivation of the pathogens is provided.

An improved photo illumination of a body fluid is provided according to an aspect of the present invention wherein a predetermined geometry of a fluid conduit is arranged to provide uniform mixing of the fluid. The mixed fluid is uniformly exposed to a photo-inactivation light source during the mixing.

A photo inactivation apparatus for a contaminated fluid containing pathogens to be inactivated according to an aspect of the present invention comprises a first fluid static mixing device having at least a partially transparent wall portion for receiving a flowing first fluid comprising the contaminated fluid including a photo inactivation agent mixed therein, the device having a predetermined geometry to continuously expose substantially all of the mixed first flowing fluid therein to the transparent wall portion.

A light source applies incident light to the mixing device transparent wall portion and to the exposed first fluid to activate the pathogen inactivation agent throughout the fluid to thereby inactivate substantially all the pathogens in the first mixed fluid.

In a further aspect, the predetermined geometry includes a plurality of stationary mixing elements serially arranged in at least a partially transparent conduit for continuously stretching, folding and interchanging different layers of the fluid during the exposure to the incident light so that substantially all of the fluid receives sufficient illumination from the light source during the exposure for the inactivation.

In a further aspect, the predetermined geometry is provided by an apparatus for continuously interchanging different layers of the fluid sample exposed to the light to assure that all parts of the fluid receive the same total illumination during the period of light exposure.

A static mixer for optical inactivation of pathogens in a fluid flowing in a conduit according to another aspect of the present invention comprises a first at least partially optically transparent conduit for receiving a flowing fluid containing pathogens and a first static mixer in the conduit forming a first mixing device having a fluid output, the first static mixer comprising a series of first helical elements, wherein each element comprises first and second helical sections of different relative orientations for exposing the flowing fluid substantially uniformly to incident light for the inactivation.

In another aspect, the conduit defines a longitudinal axis, the sections each having a given angular orientation relative to the axis, alternate sections having the same angular orientation.

In a further aspect, all of the elements are identical and each element comprises a pair of helix sections rotated relative to each other about the longitudinal axis.

In a further aspect, at least a portion of the elements are optically transparent at the optically transparent portion of the conduit.

In a further aspect, adjacent helix sections are in reverse mirror image relation.

In a further aspect, the elements are coaxial about the axis, adjacent sections are each rotated 90° relative to each other about the axis, alternate sections having the same angular orientation about the axis.

In a further aspect, the mixer sections each comprise sheet material with a helical portion channel on opposite sides of the material.

In a further aspect, the first and second helical sections are in reverse orientation and each section comprises a 180° segment of a helix.

In a further aspect, the first and second sections have a given relative angular orientation to each other about a common axis defined by the conduit, alternate sections of adjacent elements having the same angular orientation relative to the axis.

In a further aspect, a pump is included for pumping the flowing fluid into the conduit.

In a further aspect, the mixer comprises a second conduit fluid output coupled to an input of the first conduit and a second static mixer in the second conduit forming a second mixing device. The second mixer comprises a series of second helical elements. A first input to the second conduit is for introducing a first fluid containing at least one pathogen. A second input to the second conduit is for introducing a photo inactivation second fluid having a given absorbance for inactivation of the of the at least one pathogen in the first fluid in response to incident light applied to the first mixer. A first pump is for flowing the pathogen containing first fluid into the first input and a second pump is for flowing the photo inactivation second fluid into the second input.

In a further aspect, a control operates the first and second pumps at predetermined relative flow rates.

In a further aspect, a plurality of LEDs are optically coupled to the first static mixer for activating the photo inactivation fluid.

Preferably, the LEDs have at least one wavelength and intensity.

Preferably, a plurality of arrays of first conduits and static mixers are fluidly connected in one of/or both of series and parallel.

In a further aspect, a control varies the flow rate of the pathogen containing first fluid and photo responsive second fluid, light intensity and array format of the first and second static mixers according to time constraint, chemical activation requirements and total volume of the fluid to be processed.

In a further aspect, a plurality of first static mixers and mating corresponding conduit pairs are included, each latter pair forming a conduit section of a given length, a plurality of light emitting diode (LED) arrays are included, each array for illuminating a length of a different first static mixer section.

In a further aspect, a control selects the radiant power levels of the LEDs to match the absorbance characteristics of the photo inactivation fluid.

Preferably, the first static mixer comprises a plurality of static mixers and mating conduit pairs, each pair having a given length, the LEDs being located along the given length with each LED applying a discrete beam of radiant energy to the corresponding static mixer pair.

n a further aspect, the first fluid and each static mixer comprises a plurality of respective elements, the power level of each discrete beam has a value wherein the total energy absorbed as each fluid element passes through each static mixer element is the sum of the individual LED beam energies and is equal to the required energy in joules/cm\hat{ }2 to activate the photo inactivation fluid.

In a further aspect, the first and second pumps are each a peristaltic pump for flowing the respective fluids at a respective flow rate to produce a predetermined concentration of the photo inactivation fluid after mixing to a virucidal or bacteriocidal level pre-illumination.

In a further aspect, the fluid output of the first static mixer device includes photochemicals and endotoxins, further including a resin column coupled to receive the fluid output for removal of the photochemical and endotoxins resident in the fluid output.

In a further aspect, the resin column comprises a static mixer packed with the resin.

In a further aspect, the fluid output of the first static mixer device includes photochemicals and endotoxins, further including a further mixer device coupled to the fluid output for removal of the photochemical and endotoxins resident in the fluid output, the further mixer device comprising a coated static mixer in a conduit wherein the coating comprises a resin attractant material for the photochemical and endotoxin.

A method according to another aspect is for inactivation of pathogens in a fluid comprising flowing the fluid containing a photo inactivation agent through a static mixing device comprising a housing that has at least a partially transparent portion containing a static mixer while exposing the transparent portion to incident light for activating the agent to inactivate the pathogens.

A method according to another aspect of the present invention for filtering a body fluid containing inactivated pathogens comprising filtering the fluid with a static mixing device containing filtering materials.

A filtering device for filtering a flowing body fluid containing inactivated pathogens and an inactivation agent according to an aspect of the present invention comprises a static mixing device for receiving the flowing fluid and

filtering materials in the device for filtering the inactivated pathogens and contaminants.

### BRIEF DESCRIPTION OF THE DRAWINGS

FIG. 1 is a plan view of an apparatus for inactivating pathogens in a fluid according to a preferred embodiment of the present invention;

FIG. 1a is a fragmented end sectional view of FIG. 1 taken at lines 1a-1 a;

FIG. 2 is a front elevation view of a portion of the apparatus of FIG. 1 showing the light generating devices used with the embodiment of FIG. 1;

FIG. 3 is a side elevation view of the apparatus of FIG. 1;

FIG. 4 is a fragmented elevation sectional view through a static mixer of FIG. 1;

FIG. 4a is a top plan sectional view of the static mixer of FIG. 4 taken at lines 4a-4a;

FIG. 5 is an isometric view of a representative mixing element portion of the static mixers of FIGS. 1-5;

FIG. 6 is a diagram of the fluid flow constituent parts of the apparatus of FIG. 1;

FIG. 7 is a flow chart of the central processing unit (CPU) and related components operated by the CPU;

FIG. 8 is a plan view of a second embodiment of light generating devices used with the embodiment of FIG. 1;

FIG. 9 is a side elevation view of the devices of FIG. 9;

FIG. 10 is a schematic diagram of the circuit for operating the devices of FIGS. 8 and 9;

FIG. 11 is a plan view of an alternative embodiment of the static mixer array;

FIG. 12 is an elevation fragmented section of a static mixer according to a further embodiment of the present invention;

FIG. 13 is an elevation fragmented section of a static mixer according to a still further embodiment of the present invention; and

FIG. 14 is a sectional elevation view of a further embodiment of the present invention.

### DEFINITIONS

Biological or body fluid—A biological or body fluid includes any treated or untreated fluid associated with living organisms, particularly, but not limited to blood, including whole blood, warm or cold blood, stored or fresh blood, such as blood diluted with at least one physiological solution, including, but not limited to saline, nutrient, and/or anticoagulant solutions, blood components, such as platelet concentrate (PC), platelet-rich plasma (PRP), platelet-poor plasma (PPP), platelet-free plasma, plasma, fresh frozen plasma (FFP), components obtained from plasma, packed red cells (PRC), transition zone material or buffy coat (BC), analogous blood products derived from blood or a blood component derived from bone marrow, red cells separated from plasma and suspended in physiological fluid, and platelets separated from plasma and suspended in physiological fluid. The biological fluid may be treated to remove some of the leukocytes before being processed according to an aspect of the present invention. Blood products includes products similar to those described above or biological or body fluids obtained by any means with similar properties to the products described.

Unit—The quantity of biological or body fluid from a donor or derived from one unit of whole blood. It also refers to the quantity drawn during a single donation. Typically, the volume of a unit varies, the amount differing from patient to patient and from donation to donation. Multiple units of some blood components, particularly platelets and buffy coat, may be pooled or combined, typically by combining four or more like units.

Inactivation Agent—This includes at least one photo inactivation agent and a carrier fluid, e.g., a pharmaceutical carrier such as, for example, saline or sterile buffer. The inactivation agent can include a plurality of different inactivation agents. Alternatively or additionally, the agent includes a plurality of inactivation agents used together (e.g., as a mixture) or sequentially. Typical inactivation agents include, but are not limited to, photo active agents such as photo active dyes and/or their byproducts, e.g., psoralens such as methylene blue, pthalocyanine-containing agents (e.g., pc4 and pc5) and porphyrin-containing agents. Suitable inactivation agents include, for example, those disclosed in U.S. Pat. Nos. 5,166,197, 5,637,451, 5,763,602, and 5,585,503 all incorporated by reference herein in their entirety.

An inactivation agent may include liposomes, and at least some portion of a photo active agent associated with some of the liposomes. The inactivation agent may also include additional components such as, but not limited to, at least one of a buffer, a quencher and an antioxidant.

Alternatively or additionally, the additional components may be added to the biological fluid before the inactivation agent is placed in contact with the biological fluid. The additional components also may be added to the biological fluid after the biological fluid is placed in contact with the inactivation agent(s).

Pathogen—A pathogen includes, but is not limited to viruses, (both encapsulated and naked), bacteria, yeasts, fungi and parasites.

Inactivation—killing and/or removal of pathogens.

Contaminants—All matter that is foreign to and which may be added to a body fluid.

Additive to the testing of all blood units involved in patient transfusion, both of whole blood as well as blood derivatives such as platelets, plasma and other blood components has been the efforts to develop a way of inactivating virtually all pathogens in blood units.

Researchers have been working on chemical inactivation, filtration techniques and photo-inactivation of pathogens to rid transfusion fluids including blood products of unsafe vectors.

This invention is concerned with an improved method and apparatus for the effective inactivation of pathogens in blood products using many of the available photo-activated agents.

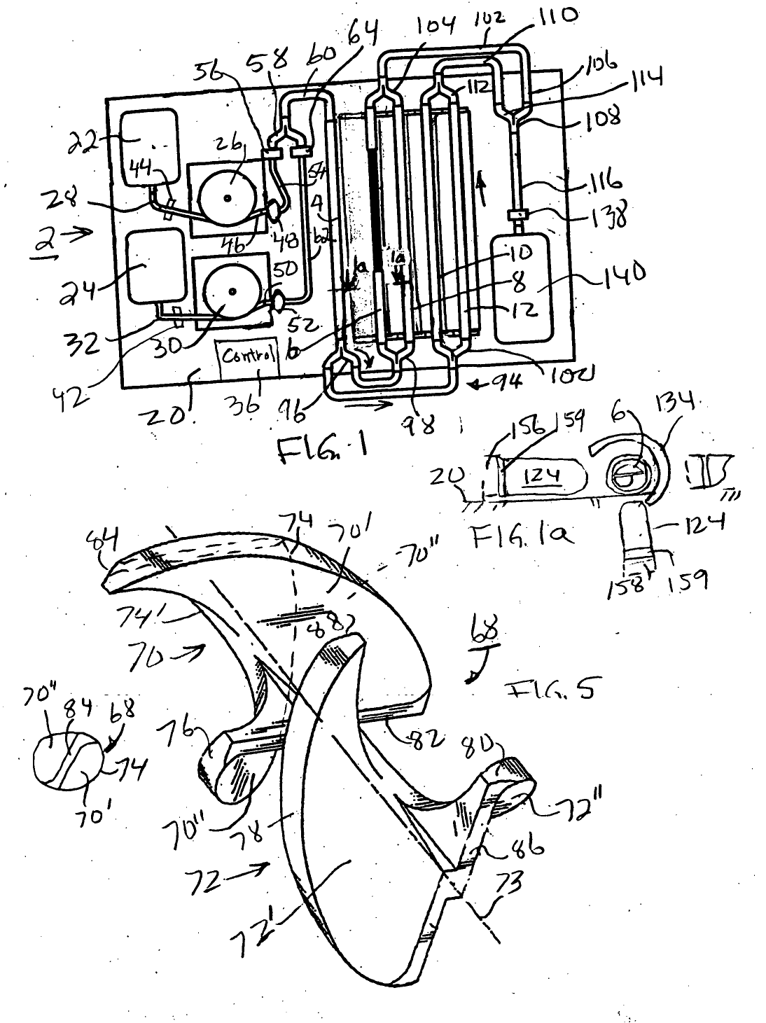

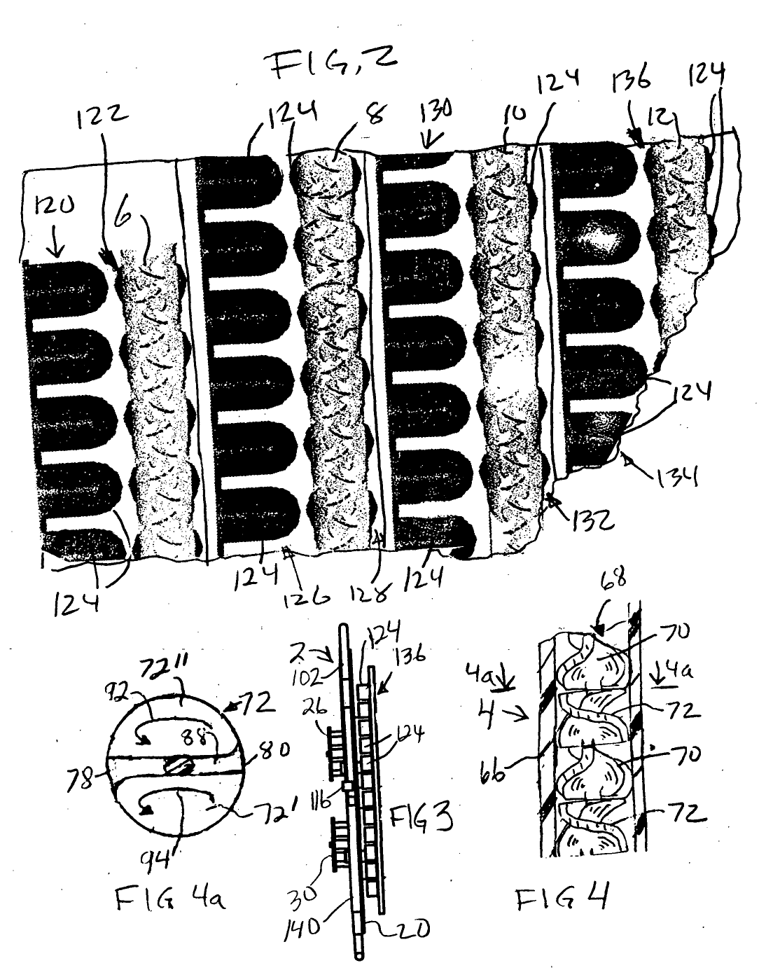

In FIG. 1, apparatus 2 is secured to a support 20, a frame, which may be a flat board or plate. A body fluid container 22 is releasably attached to the support 20 by a hook or the like (not shown). The container 22 is a disposable flexible bag such as a conventional I.V. bag or other pliable plastic or elastomeric or other material container for the fluid or a rigid container. It may or may not be opaque according to a particular implementation. A disposable photo-inactivation agent container 24 of the same or similar material as container 22 is also attached to the support 20 in similar fashion.

The body fluid in container 22 is applied to a peristaltic pump 26 via preferably disposable plastic tubing 28. The photo-active agent in container 24 is applied to peristaltic pump 30 via preferably disposable plastic tubing 32 in parallel to tubing 28. Peristaltic pumps are commercially available such as provided by the Cole-Parmer Instrument Company, Vernon Hills, III or other suppliers. The flow rate of the fluid pumped by the pumps is variable and controlled by a programmed central processing unit (CPU) 34 (FIG. 7) in control 36. The CPU 34 includes a programmable ROM 38 and RAM 40 for providing detailed operating instructions to the pumps according to a given implementation as determined for a given set of parameters of the fluids in the containers 22, 24, e.g., the volume and viscosity of the fluids being processed, the number of mixer devices such as devices 4-12, filters employed and other variables, as determined empirically for each implementation. The ROM 38 and RAM 40 are programmed with instructions accordingly.

The flow rate of the pumps is also determined accordingly to provide optimum exposure of the fluid to the inactivation illumination radiation to be described below. Manual shut off valves 42 and 44, which preferably may be disposable pinch valves for the pliable tubing 28 and 32, cut off the flow of fluids from the respective containers 24 and 22, FIG. 1, to the pumps 26 and 30. In FIG. 6, the respective photo inactivation reagent agent is in container 24′ and blood is in container 22′, for example.

Examples of photo inactivation agents include:

Methylene Blue

Endogenous compounds (naturally occurring found in the body) such as:

Allaxozines

K- and L-vitamins

Protoporphyrin

Chlorpromazine

Riboflavin

Napththoquinones, naphthalenes, naphthols and their derivatives

In addition, the photo active agents maybe derived from a family of light activated drugs derived from benzoporphyrin commonly referred to as BPD's. These are commercially available from Quadra Logic Technologies, Inc. Vancouver, B.C., Canada. These, like the above mentioned types of agents and other types of hematoporphyrin agents, have an affinity for cell walls of many viral organisms carried in the blood. The agents bind themselves to the biological cell wall of the pathogens. When exposed to illumination radiation, photo inactivation agents undergo an energy transfer process with oxygen, forming a singlet oxygen molecule. When the singlet oxygen molecule oxidizes, it kills the biological cell of the pathogen to which it is attached. See for example U.S. Pat. Nos. 5,290,221 and 4,878,891, incorporated by reference in their entirety herein. Still other photo agents are known and may be used in the preferred embodiments.

The operating parameters of the inactivation agent can be adjusted to accommodate the individual agent activation specifications in a known manner. This represents a more universal approach to photo-inactivation processing of body fluids such as blood and blood products. Such operating parameters as processing time, activation light energy and wavelength requirements, operating sample temperatures and log reductions in pathogen levels can be provided by additives as known in this art.

In FIGS. 1, 6 and 7, tube 46 connects pump 26 to valve 48, which may be disposable, controlled by control 36. Tube 50 connects pump 30 to valve 52, which also may be disposable, controlled by control 36. Tube 54 connects valve 48 to flow sensor 56, which also may be disposable, coupled to control 36 (FIG. 7). The sensor 56 is coupled to the inlet of disposable static mixer device 4 by one leg of Y-shaped pipe 58 and U-shaped pipe 60 both of which preferably are disposable plastic tubing as is all of the tubing and pipes used in the apparatus 2. Tube 62 connects valve 52 to flow sensor 64, also which may be disposable, coupled to control 36 (FIG. 7), and is coupled to the inlet of static mixer device 4 by the other leg of Y-shaped pipe 58 and U-shaped pipe 60

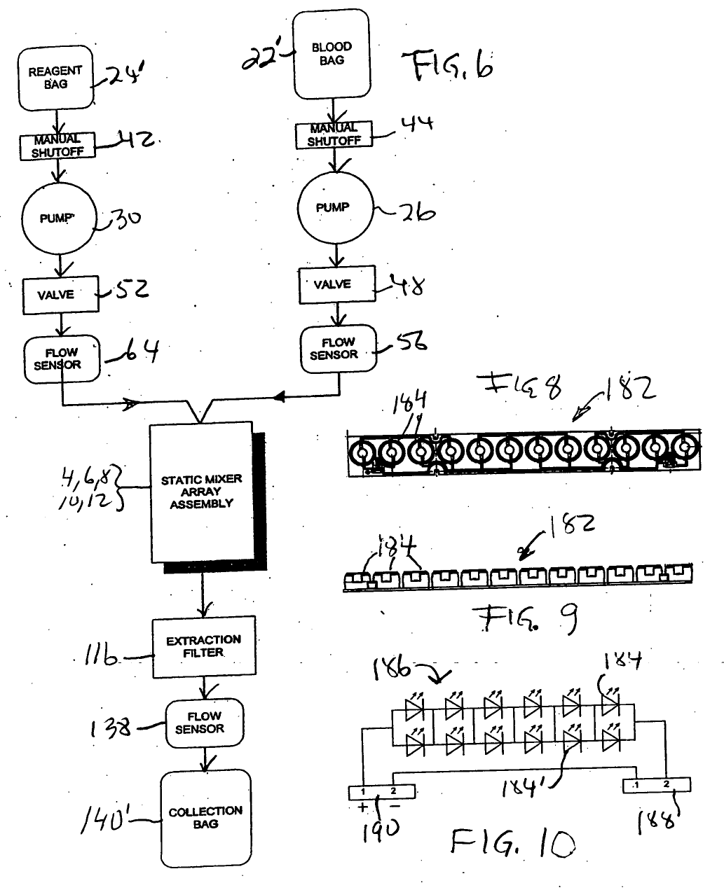

Static mixer device 4, FIG. 4, comprises a predetermined length of tube 66, which may be rigid or flexible tubing, transparent or opaque. Inside of the core of the tube 66 is a length of static mixer elements 68. The tube 66 and elements 68 may be transparent or opaque. FIG. 5 is a detailed drawing of a static mixer element 68 used to thoroughly mix different fluids and/or chemicals in fluid form (gaseous, powder or liquid).

Each of the elements 68 comprise a serial array of alternating two twisted mixing sections 70 and 72, FIG. 5, coaxial on longitudinal axis 73. Each section is a 180° turn of a helix. Section 70 is oriented in a reverse helix orientation from section 72. In addition, section 72 is rotated 90° counter clockwise relative to section 70 relative to axis 73. Each section preferably has a helix curved surface 70′ and 72′ on one side, respectively. The two mixing sections repeat in the same orientation on the same element a number of repetitions, e.g., 12 times in one embodiment, where the series of 12 repetitions of elements 68 are about 20.3 cm (about eight inches) long. Section 70 terminates at spaced helix edges 74, 76 and section 72 terminates at spaced helix edges 78, 80. Opposite surface 70′ is helix surface 70″. Opposite surface 72′ is helix surface 72″. The opposing surfaces are formed by a helix shaped plastic molded sheet member.

The section 70 opposite helix surfaces 70′, 70″ terminate somewhat normal to and at lower straight edge 82 between edges 74 and 76. Similarly the surfaces terminate at an upper straight edge 84 parallel to and axially aligned over edge 82 in the direction of the axis 73. The edges 82, 84 are normal to and lie on the longitudinal axis 73. The section 72 opposite helix surfaces 72′ and 72″ terminate at edges 78 and 80. Surfaces 72′ and 72″ terminate at lower straight edge 86 and at upper edge 88, which is parallel to lower edge 86. Edges 86, 88 are oriented 90° from the orientation of edges 82, 84 in the counter clockwise angular rotation about axis 73.

Static mixers have been in use in industry for almost thirty years and that, to the knowledge of the present inventors, have not been used for body fluids. In realizing the nature of the problem with present optical inactivation systems, the present inventors investigated such static mixers in respect of body fluids. Static mixer devices using a mixer such as mixer 68 construction have been in industrial use for several decades in a number of configurations. Virtually all versions of the static mixers share a common mixing method. However, in accordance with the present invention, the static mixer 68 may be optionally transparent when used for photo inactivation as discussed hereinbelow. Prior art mixers and mixing devices are opaque.

The present inventors have found that static mixers are effective for mixing fluids of various types from aqueous solutions to slurries. They are used extensively in the paint industry, for large scale blending of many industrial agents. Available mixer diameters vary from several feet to less than 2 mm. They are commonly used as epoxy and other adhesive applications to assure uniform consistency and mixing and are typically opaque. Static mixers are not known to be used with blood, much less with photo-inactivation processes in connection with inactivation of pathogens in body fluids.

Static mixer devices employ a housing or conduit in which is located a static mixer comprising a series of static mixing elements arranged in a serial array along a fluid flow axis in the conduit. Static mixers for such devices as described above have a series of helical mixing elements which reverse twist at each successive element of the mixer. “Mixing in the elements occurs through a combination of flow splitting and shearing at the junctions of successive elements and a stretching and folding mechanism within the elements, making the static mixer an excellent radial mixing device”. (from ‘Laminar Flow in Static Mixers with Helical Elements—Andre’ Bakker, Richard D. LaRoche and Elizabeth M. Marshall) Online CFM Book at http://www.bakker.org/cfm.

Static mixers are available in molded thermoplastic materials in small diameters from several vendors. Molded plastic helical mixing elements are commercially available in sizes suitable for insertion into glass, clear plastic or medical grade tubing of the appropriate diameter. The mixers fit in the tubes in close sliding fit to form a mixing device, but are neither too loose such that fluid bypasses the mixers in the tube or in interference fit, which might otherwise distort the mixers, in the optimum application.

These mixers require only the energy of the fluid flow to operate and do not move within the tube, hence the term “static.” Placed in a moving circularly constrained fluid stream, the stream flow becomes ovoid or elliptical as shown by arrows 92, 94, FIG. 4a. These devices “fold” the flow with optimum efficiency over relatively short distances. Each element section pair, such as sections 70 and 72, FIG. 5, provides a total “double” fold and has a mixing efficiency of 2N in small cross sections (for example 6 mm). A flowing blood mixture with a 16-elements is mixed many thousands of times over a distance of several centimeters (inches) e.g., 25 centimeters. Depending on static tube size, length and diameter, and element count, dispersion within a static tube array of the photo-agent in the mix of blood is precisely calculable and repeatable.

As a fluid sample passes through the static mixer, mixing in the elements occurs through a combination of flow splitting at the junctions of successive elements and a stretching and folding mechanism within the elements. With each passage through one of the static mixer elements, there is a binary division of the flow cross section. The number of subdivisions as a liquid element traverses a complete static mixer is equal to 2\hat{ }ⁿ, where n equals the number of helical elements in the length of static mixer.

The stretching and folding mechanism interchanges internal layers of the liquid sample with external layers of the sample. When used in a photo-illumination application, the action of the static mixer on fluid flow assures that all parts of the flowing sample are exposed to substantially the same illumination levels, providing homogenous, repeatable results. The geometry being fixed, there are no random illumination events which affect reproducibility.

The chart Table 1 shows the interaction of flow, mixer diameter and length, light source levels and characteristics.

TABLE 1
Summary of Various Combinations to Achieve Approximately 9
joules/cm\hat{ }2

| Static Mixer | # of LEDs per | # of sections of | | |
|---|---|---|---|---|
| Diameter | section | static mixer | joules/cm\hat{ }2 | total LEDs |
| 0.1875 | 6 | 16 | 9.134 | 96 |
| | 8 | 12 | 9.134 | 96 |
| | 12 | 8 | 9.134 | 96 |
| 0.25 | 6 | 12 | 9.134 | 72 |
| | 8 | 9 | 9.134 | 72 |
| | 12 | 6 | 9.134 | 72 |
| 0.369 | 6 | 8 | 8.99 | 48 |
| | 8 | 6 | 8.99 | 48 |
| | 12 | 4 | 8.99 | 48 |

* - Note:
The chart applies whether the static mixer sections are placed in parallel or series.

Choice of diameter depends upon entrapment of air and other physical parameters.

A static mixer device as used herein preferably refers to the combination of the series of elements 68 and housing or tube containing the elements forming a fluid conduit. The elements 68 may be all opaque or may have a transparent portion at the region where exposed to incident light radiation. Preferably the elements are transparent at at least this region. The tube or housing forming the conduit for the mixer device and fluid flow, however in all cases, is at least partially transparent at that portion of the mixer to be exposed to incident light. However, it should be understood that mixing elements of other constructs may be used according to a given implementation.

From a photometric dosage standpoint, a static mixer device fabricated from materials having the transparency as described above operated within a fixed light field will subject the mixture flowing therein to a predictable and consistent photon flux during passage through the fixed-distance, short-path, high-efficiency optical geometry. The fluid flowing in the mixer device 4 induced by the peristaltic pump comprises the photo-inactivation agent from container 24 and the pathogen contaminated body fluid from container 22. The mixer device 4, however, since it mixes these two fluids does not need a transparent portion as it is not exposed to light. The device mixes the two fluids to an optimum mixture. The device 4 may preferably have a mixing diameter preferably in the range of 4 to 18 millimeters and preferably a mixing length in the range of 15 to 30 centimeters with respect to mixing blood or plasma with a photo-inactivation agent. However, these values are exemplary only, as such values depend upon a given implementation and may be best determined empirically. For these fluids the pump should preferably produce a flow rate of about 7.5 ml/min or in the range of 7.5 ml/min. to 15 ml/min max over this length of a mixer device to produce optimum mixing.

As shown in FIG. 1, an array of parallel elongated static mixer/tube devices 6, 8, 10, and 12 are connected in fluidic parallel flow to the body fluid and photo-inactivation agent mixer device 4. These devices comprise at least a partially transparent tube portions and a series of preferably at least partially transparent mixer elements such as elements 68 which are transparent in the region where exposed to incident light. The mixer elements may be transparent to obtain optimum efficiency. It should be appreciated that depending on the location of the light source, these elements may be opaque as long as the housing or conduit containing the elements is transparent at least in the region where the light is required to be incident on the mixed flowing fluid. However, by making the mixing elements transparent, maximum incident light is received by the flowing fluid in the flow. Flow is split equally into each of four devices 6, 8, 10 and 12 from supply device 4 by tubing arrangement. The arrangement 94 comprises Y connector tube 96 which splits the flow evenly to Y connector tubes 98 and 100 all of which tubes are equal inside diameter disposable plastic tubing. Flow of the fluid in the tubes is in the direction of the adjacent arrows. Each of the static mixer devices 6, 8, 10 and 12 have transparent portions in the light exposed regions, and are identical in length, diameter and configuration of the mixer elements inside of the corresponding tubes. The devices 6, 8, 10, and 12 preferably are all transparent tubes and include transparent mixers of the same length and number of mixing elements 68 inside the core of the tubes. Each of the devices 6, 8, 10, and 12 is exposed to electromagnetic radiation in the form of pathogen inactivating light emitted by adjacent arrays of light emitting diodes LEDs, e.g., at approximately 635 nm (red) or 450 nm (blue). Single, double or triple rows of LEDs are possible depending upon the required parameters of light energy and throughput time.

In FIG. 2, portions of mixer devices 6, 8, 10 and 12 are shown in more detail with the adjacent light emitting diodes. Adjacent to transparent mixer device 6 are two arrays 120, 122 of identical diodes 124. Array 120 illuminates device 6 from the side and array 122 illuminates device 6 from the rear side into the drawing sheet (and thus are only shown partially). Similarly, arrays 126, 128 illuminate mixer device 8, arrays 130, 132 illuminate device 10 and arrays 134, 136 illuminate device 12.

To assist in maximizing illumination of the static mixer devices 6, 8, 10, and 12, a reflector 134, FIG. 1a is located over the LEDs 124. A second reflector 136 is over the LEDs 124 in the rear. The reflectors may be coated with optical spheres to enhance reflectivity and the reflectors shown are representative and may differ in configuration from that shown and described, which is by way of example only. Other reflective embodiments utilizing highly polished mirror or other reflective known surfaces may also be used.

The light intensity profile can be linear for a linear light source, such as an arc, or, be present in a series of energy packets produced by a series of discrete light sources such as the above described LEDs. Other high energy discrete light sources may be used in the alternative to the arrays of light emitting diodes (LEDs), e.g., incandescent lamps, arcs or fluorescent lights.

A cooling device may be used to remove heat from the LEDs in order to maintain the semiconductor diode temperatures at the appropriate temperature, e.g., near room temperature (25° C.), so as to maintain the rated operating life of the LEDs and rated light power output. In FIG. 1a, for example, thermoelectric cooling devices 156 and 158 (shown in phantom) under control of control 36 (FIG. 7) are attached to the base of diodes a24 via a cooling metal plate 159 to cool the arrays 120, 126 and so on, FIG. 2. A curved reflector 134, e.g., parabolic, is on the side of the mixing device opposite the LED, such as device 6, for example, to reflect light back to the device. The reflector 134 for example reflects light to the LEDs 124 in two different, e.g., orthogonal, orientations.

In the alternative, a heat sink, i.e., plates 156 and 159, may be extended and cooled by forced air (not shown) may be used. The plates are thermally conductively attached to the thermally conductive portions of each diode of the diode arrays, FIG. 2.

In a further alternative embodiment, the thermoelectric devices 156 and 158 may be thermally conductively attached to a cold thermally conductive plate (not shown) in intimate thermal conductive contact with the bases of the LEDs for efficient heat removal.

In the alternative, the temperature control may elevate the fluid temperature if such elevated temperature enhances activation of the pathogen inactivation agent.

The mixer devices 6 and 8 are coupled to a tube 102 by an output Y connector tube 104. Tube 102 terminates at one leg 106 of Y connector tube 108. The mixer devices 10 and 12 are coupled to a tube 110 by an output Y connector tube 112. Tube 110 terminates at the other leg 114 of Y connector tube 108. Tube 108 serves as an inlet for the mixed pathogen optical inactivated fluids to extraction tube and filter 116.

The flow rate through each device 6, 8, 10 and 12 is equal to one fourth of the total flow in supply device 4, by way of example. See Table 1 which shows different combinations of elements and LEDs to obtain about 9 Joules/cm\hat{ }2. This flow rate and volume is calculated to process the flow volume of a body fluid sample/photo-inactivation chemical mixture in a specified processing time. These values can be determined empirically for each implementation. Device 4 mixes a photo-inactivation agent supplied in container 14 with the body fluid supplied in container 16 as described above and thus is not exposed to light, but may be.

The flow rate through each leg is such that one fourth of the sample absorbs the applied radiant energy in a predetermined specified time. This can be expressed in joules/cm\hat{ }2. The energy absorbed is a function of the total radiant energy impinging upon each static mixer 6, 8, 10 and 12, the mixer diameters and the flow rate. As a body fluid such as a blood element passes through one static mixer channel, it absorbs an amount of radiant energy during its passage dependent upon the velocity and diameter of the tube forming the volume of fluid flowing through the static mixer and the light intensity on the fluid. The wavelength and radiant power are preferably selected to match the absorbance characteristics of the photochemical inactivation agent/fluid mixture.

A filter 116 is connected to a flow sensor 138 which outputs the filtered cleansed fluid into the collection container 140 which preferably is a disposable pliable plastic bag or other collection container as presently commercially available. The filter 116 removes all contaminants and inactivation agents.

FIG. 1 depicts one embodiment of a processor for the photo-inactivation of a blood sample in which the static mixer device array and all of the lines, fittings, sample supply bag container 22, photo-inactivation chemical removal filter 116 and final product bag container 140 are incorporated into a sterile, disposable apparatus, except for the pumps, and optionally the sensors and valves such as valves 48 and 52 which may remain fixed in place or, in the alternative, which may also be disposed of. The photo-inactivation removal arrangement in one embodiment comprises a filter 116 which is preferably contained in a tube. In the alternative, a resin column or a static mixer/resin filtering device or a static mixer coated with a contaminate removal material located in a tube or flow housing may be used.

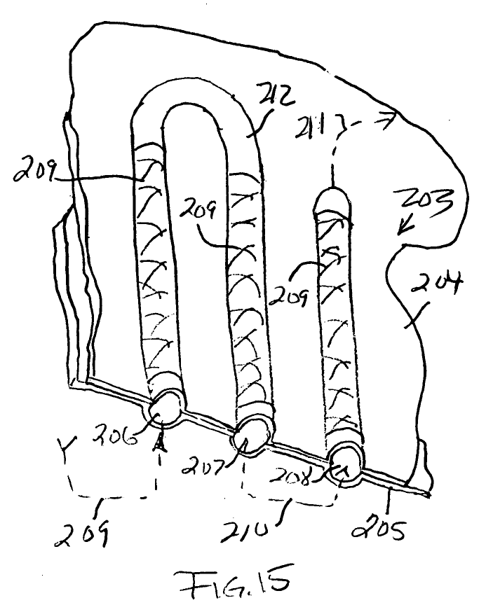

In FIG. 12, filter 116 of FIG. 1 comprises a static mixer device constructed of a static mixer 144 as described above in connection with FIG. 5 located inside a tube 146. These may be opaque. Packed inside of the tube 146 and about the mixer 144 are resin particles 148. The particles 148 are resin adsorbents which effect removal of the photochemcials comprising the photo-inactivation agents and endotoxins resident in the treated fluid.

Resin adsorbents are a series of products based ion-exchange resin manufacturing technology. Synthetic resin adsorbents are a series of products based on ion-exchange resin manufacturing technology, and are designed for use as solid particle extractants. These resins have large surface areas and fine interior pore structures such as in activated carbon. With this characteristic, they effectively adsorb organic compounds from solutions.

A synthetic adsorbent is a spherical particle and the effective fine pore structures are suitable for the diffusion of solutes that contain the component to be removed. Small solutes can penetrate the particle by diffusing through the pores. In contrast, molecules that are larger than pore size cannot penetrate into the inside of the particle. Consequently, such molecules are not adsorbed on synthetic adsorbents, resulting in so-called molecular sieving. The most appropriate type of adsorbent can be selected according to the pore size of the adsorbent and the molecular size of the target compound (or sometimes unfavorable compounds to be removed). Another important parameter is the surface area of resins. Adsorbents with large surface area shows high uptake capacity, especially for small molecules

Ion-exchange resins are classified based on their chemical structures or functionalities.

| | | Functional | |
|---|---|---|---|
| Type | Base | Group | Matrix |
| Strongly acidic | styrenic | sulfonic | gel/porous/highly |
| cation- | | acid | porous |
| exchange | | | |
| Weakly acidic | acrylic/methyl | acrylic acid | porous |
| cation- | acrylic | methyl | highly porous |
| exchange | | acrylic acid | |
| Strongly basic | styrenic | quaternary | gel/porous/highly |
| anion- | | ammonium | porous |
| exchange | | types land | |
| | | II | |
| Weakly basic | styrenic | tertiary | porous |
| anion- | acrylic | amine | highly porous |
| exchange | | polyamine | |
| others | | special | highly porous |
| (chelating | | ligands | |
| resins) | | | |

Typical applications of ion-exchange resins include water treatment, waste water, sugar refining, food processing, and chemical purification. Manufacturers of these various resins include Amersham Biosciences, J. T. Baker, Biochrom Labs, Inc., Bio-Rad, Ciphergen, Dow Liquid Separations, Millipore, Novagen, Purolite, Sigma-Aldrich, Tosoh Biosep, Mitsubishi Chemical and Whatman.

To determine the most appropriate resin for a particular application, screening of the adsorptive qualities of the resin in removing the desired chemical entity from the solution/suspension must be performed. This is initially done by exposing a known concentration of the compound to be removed to a specific amount of resin, allowing them to remain in contact for an established time, separating the compound from the resin, and analyzing it by an approved quantitative means to determine the residual concentration. A similar quantity of compound of interest should be assayed under identical conditions but without resin. From this, one can establish experimentally the amount of compound adsorbed per weight of adsorbent. Using this initial information, one can proceed to construct an adsorbent filter to be used in processing the solution of interest. In testing nine different resins selected on the basis of charge, over 98% of the compound examined was bound on the basis of anion-exchange.

In the case of processing a suspension such as blood, the filter must be prepared so that the red blood cells are not entrapped while the compound to be removed from the solute is able to diffuse into the resin and be adsorbed. Selection of the appropriate sized resin particles is critical to recovery of the blood cells.

The resin can be packed into a membrane having a pore size to allow the molecules in the solute to diffuse into the resin and be removed. Another means for exposure of the resin to the blood suspension is to affix the resin beads to an internal surface so that the blood cells in suspension flow over and around the resin. This surface, for example, could be the helices of the static mixer which provides extensive surface area exposure Moreover, the inner surfaces of the container in which the static mixer is positioned can also be modified so that resin can be affixed without impeding the flow of blood.

In the case where the compound is a photo-inactivation agent, it is necessary to remove this from treated blood before the blood is used for transfusion. Thus, the filtration by resin could be incorporated in a filter mixing device and the blood collected in a sterile bag following filtration. Alternatively, the filtration could occur in a stand alone filtration device; however, this is not a preferred method since additional breaks in sterility occur when utilizing more than one sterile set-up.

Endotoxin Removal:

Besides the requirement to remove the photo-inactivation compound from treated blood, there is the outside possibility that the initial blood may have been contaminated with gram-negative organisms that could release endotoxin. Endotoxin is a lipopolysaccharide (LPS) that is found in the cell wall of gram-negative bacteria and is very toxic to mammals, causing fever and shock even when injected in very small amount. It is negatively charged and hydrophobic in character. Affinity membranes have been employed to remove this contaminant and attention to the flow rate, pH, and ionic strength is necessary. There is strong interaction of the lipopolysaccharide with positively charged surfaces and selection of an appropriate material is made on this basis. Resin chromatography is one means of treating materials so contaminated. A cationic polymer can be employed, but adsorption takes place mainly from the outer surface of adsorbent particles. Resin manufacturers include Stratagene (Acticlean), Merck KgaA (Fractogel) and Sartorius (Sartobind)

In processing fluids, membrane filtration will also adsorb LPS and such membranes are commercially available in various pore sizes. For example, Pall manufactures a N6,6 Posidyne membrane. Alternatively there is available from Cape Cod, Inc. a compound that is endotoxin specific, End-X. This is a binding protein derived from the horseshoe crab Limulus polyphemus. This material can be immobilized on silica beads as an endotoxin affinity ligand.

To assure that any potential contamination by LPS in treated blood is eliminated, a resin capable of adsorbing this compound would be incorporated in the final blood filtration device. Alternatively the affinity ligand could be employed by affixing it to surfaces to which the blood suspension comes into contact. The standard testing for LPS is performed by utilizing a quantitative limulus lysate assay.

In view of the above, in the alternative, the static mixer 144 may be coated with resin particles comprising immobilized resin or other attracting material for attracting the photochemical contaminants. The efficient static mixer dynamics assures adequate turnover of fluid layers to adsorb the photochemical/endotoxins of the flowing fluid in intimate contact with the coated surfaces. This removes the contaminants passing through the column. The static mixer brings two substances into immediate contact for thorough admixture. Passage of the reactant can be controlled by a pump (not shown) so the contact time between the adsorbent and substance to be removed is optimized.

In FIG. 14, a porous membrane 152 having an anti-endotoxin coating 154 may be placed in a tube 150. Contaminant material such as an endotoxin is entrained by the anti-endotoxin coating 154. At the exit of the column is a further filter (not shown) such that biological particles of interest such as red blood cells in the suspension can exit the column without damage while particulate matter from the resin is entrapped. An example, is C-107E resin applied to the surfaces of the static mixer such as mixer 144, FIG. 12, and a fine mesh nylon filter (not shown) located downstream.

In FIG. 7, the apparatus 2, in addition to the components discussed above, includes the following components coupled to the CPU such as keypad and/or touch screen 162, a display 164 for displaying data, status and various sensor outputs, and a temperature control 166 for adjusting the cooling devices to control the temperature of the mixed fluids in the static mixers based on the temperature sensed by sensor 180. An LED intensity control 168 is set by the program of the CPU to vary the illumination intensity of the LEDs to the predetermined value and in response to the temperature sensed, and optionally also adjust the fluid flow rate, for a given implementation. Data can be stored in memory 182 from the various sensors under control of the CPU 34 for accumulating test and operating data for research and evaluation. Such data can also be used to adjust the parameters in real time by the CPU as the process is operating. A power supply 192, which may be standard household current, applies electrical power to the control 36 and related components.

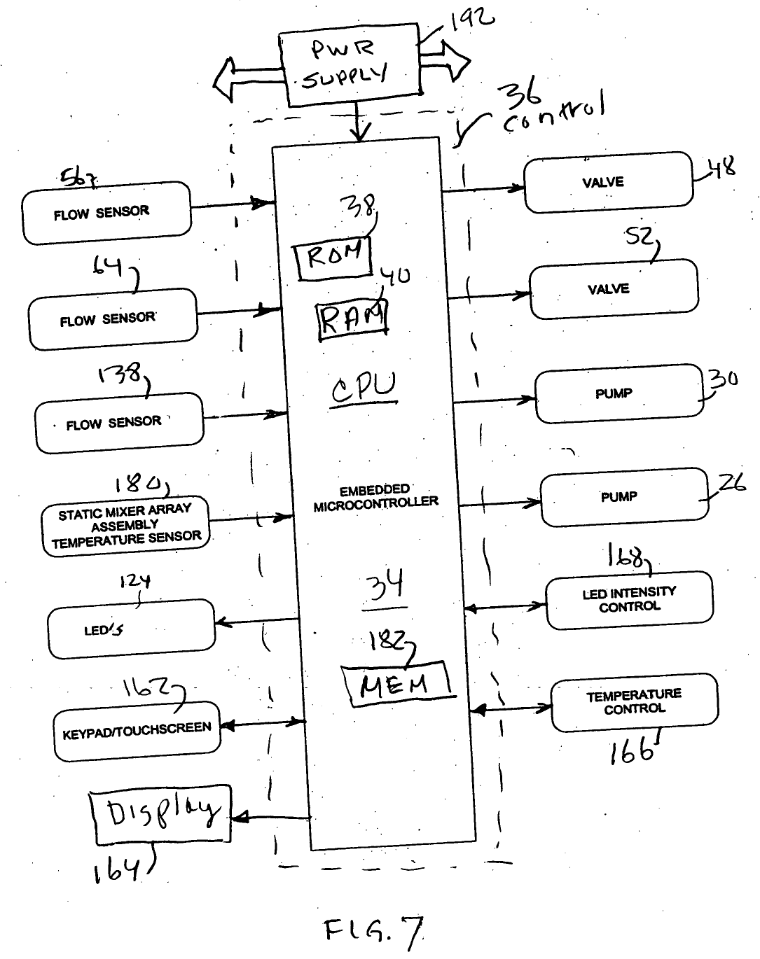

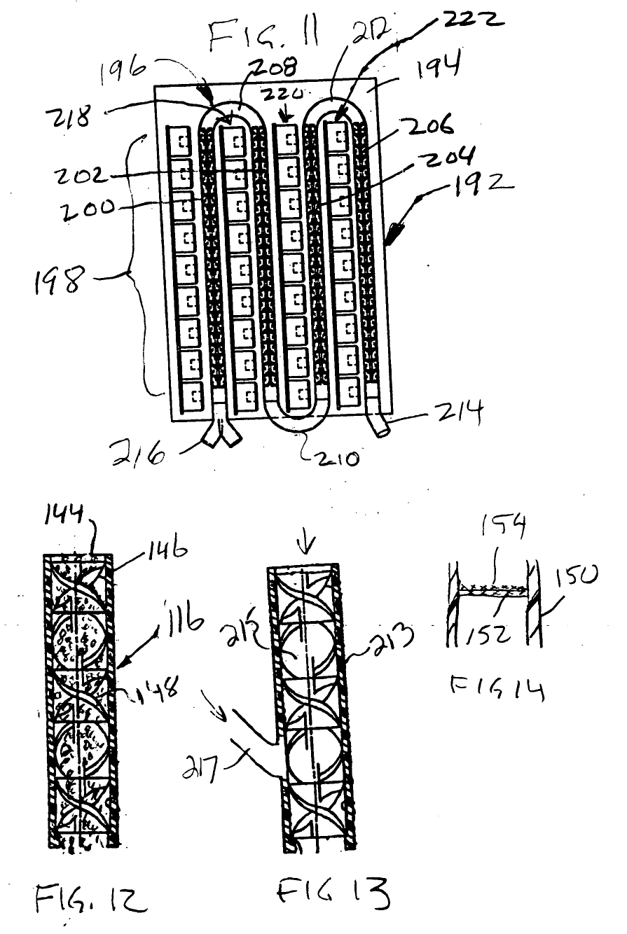

In FIGS. 8 and 9, an alternate embodiment of an array device 182 of LEDs 184 is shown. This device is available from Luxeon with the LEDs in white, green, blue, cyan, red and amber as desired. This is referred to by Luxeon as the Lexeon™ Line. The LEDs are identical and there may be 12 LEDs in the array. The LEDs are mounted on a printed circuit board with a conventional AMP connector at either end. The array is about 290 mm long and 15.7 mm high. The circuit diagram 186 is shown in FIG. 10 wherein two parallel arrays of LEDs 184 and 184′ of six LEDs each are coupled between preferably AMP 2 pin CT connectors 188 and 190, AMP type board, code 2-179123-2 and AMP housing device code 173977-2. A jumper wire (not shown) is inserted in the right hand connector 188, the left hand connector 190 for receiving power.

The arrays of LEDs 184 are assembled to the static mixer device arrangement 192, FIG. 11 in a further embodiment. The arrangement 192 includes a support 194, and a continuous serpentine tube 196. The tube 196 preferably is at least partially transparent at least in regions 198 in each portion 200, 202, 204 and 206. Tube portions 200-206 are preferably linear segments interconnected by a preferably U-shaped tube section. Tube portion 200 is connected to tube portion 202 by tube section 208. Tube portions 202 and 204 are interconnected by tube section 210 and tube portions 204 and 206 are interconnected by tube section 212. Tube 196 has an end outlet 214 which goes to a filter such as filter 116, FIG. 1 and a collection container such as container 140, FIG. 1. Tube 196 has a bifurcated inlet Y section 216 for receiving the inactivation agent fluid and the body fluid. In the alternative, the inlet Y section 216 can be replaced by a further static mixing device such as device 4, FIG. 1, for mixing the body fluid and inactivation agent.

Adjacent to each portion 200, 202, 204 and 206 is a corresponding respective LED array 218, 220 and 222 of LEDs 184. The remaining components of the system of FIG. 11 may include components such as disclosed in FIG. 1 for example.

In FIG. 13, a tube 213 includes a mixer 215 in its conduit. Mixer 215 comprises elements 68 as described above. The tube 213 is transparent and the mixer is opaque. An inlet port 217 is coupled to the tube 213 for inputting a further fluid into the tube 213. Material does not flow out of the port 217 because it is inclined upwardly and may flow fluid into tube 213 by pump pressure or by gravity, for example. Also the fluids may be derived from reconstituted powders as well as liquids as flowing in the mixers described herein.

In another embodiment of the mixer devices, a tube assembly 203, FIG. 15, comprises two mating sheet material sheet member shells 204, 205, preferably molded sheet plastic material, of mirror image shape. The shells 204 and 205 are bonded together to form leak proof channels 206, 207 and 208 and so on in which the elongated helical mixing elements 209 are disposed. The channels have partially transparent wall portions. The channels 206, 207 and 208 are interconnected in a continuous (or parallel as desired) flow path by channel 212 and other channels formed in the shells 204 and 205 as represented by the dashed lines 209, 210, 211. The particular configuration of the channels, the number of mixing elements per channel, the flow arrangement and so on is according to a given implementation. The assembly 203 thus formed is disposable. Not shown are valves, sensors and other devices that may be molded integral one piece with the shells 204, 205 or assembled thereto via specially molded cavities in the shells (not shown). The entire assembly 203 with such devices, sensors, valves and so on may be disposable.

### EXAMPLE

A static mixer diameter of 9 mm was chosen and the LEDs were placed adjacent to the static mixer tube such that the inner beam diameter matched the static mixer diameter. In this example, a static mixer device was tested for the inactivation of bacteria in a blood sample. Packed blood cells at a hematocrit of about 60% were pumped through a 9.53 mm (3/8 inch) diameter, 3.05 cm (12 inch) long static mixer device. The blood had been previously seeded with 10\hat{ }7 cells of Pseudomonas aeruginosa and a concentration of a porphyrin derivative mixed with the blood.

The blood flow was approximately 0.0375 ml/sec through the static mixer device. A series of high energy LED light sources at about 645 nm wavelength and 135 mw radiant energy output were in close optical coupling with the static mixer. Post illumination, the bacterial counts were reduced greater than 5 logs to below 10\hat{ }2 cells in the whole sample.

This technique can be used to inactivate contamination in many different biological samples using any one of many photoactivation agents. Light level, wavelength and flow can be adjusted for optimal pathogen kill based upon the characteristics of the chosen agent which can be readily determined by one of ordinary skill in this art.

In the apparatus of FIG. 1, the photo-inactivation agent can be premixed with the body fluid sample or mixed on line in a separate static mixer device (not shown) prior to entering into the illumination section. The photo-inactivation agent can be mixed with the biological sample in liquid form, or can be reconstituted from a dry form by either the body fluid or other fluid sample under test or a supplied diluent.

If necessary, the photo-inactivation agent can be removed by a number of adsorption filtering arrangements including those described above.

The highly efficient static mixed fluid dynamics assures that there is adequate turnover of fluid layers to adsorb the photochemical/endotoxins of the flowing sample in intimate contact with the coated surfaces. The use of a static mixer entrained in a column the diameter of which makes a tight fit with the radially exterior edges of the wings of the mixer allows the device to be employed to remove substances from the liquid/suspension passing through the mixing device column. Conversely, the static mixer device can be employed to bring two substances into immediate contact for thorough admixture.

When used as described in the first instance, i.e., to remove substance(s) from material passing through the mixing column, the adsorbent may be bound to the surfaces of the static mixer in the device and/or the inner portion of the surrounding mixer column. Passage of the reactant can be controlled by a pump so that the contact time between the adsorbent and substance to be removed is optimized. An adsorbent resin can be placed in a mixer column so that it is present on the surfaces of the wings of the static mixer elements and/or on the interior of the column. As the liquid/suspension flows through the column, adsorption of the substance to be removed occurs.

There thus has been shown that relatively high power light emitting diodes (LED) provide the illumination, which diodes do not generate undesirable elevated heat values. These diodes thus eliminate the heating problem inherent in most prior art systems. LEDs are efficient in respect of converting electrical energy to light and in their ability, because of their small geometry, to be placed physically close to the sample being processed.

Also there has been shown that to provide for continuously interchanging different layers of the fluid sample exposed to the light, static mixers are inserted into at least one optically transparent tube for receiving the fluid being processed.

It has been further shown that at least one tube is provided a length and volume and the body fluid is flowed in the at least one tube at a predetermined flow rate. This exposes the fluid in the tube for the predetermined time period to inactivate substantially all of the pathogens in the fluid.

LEDs have an important advantage over other types of light sources in that when operated at rated temperatures, their operating life is relatively long, e.g., measured in tens of thousands of hours.

Thus as shown, the use of static mixers efficiently solves the inherent problems present in current methods of pathogen inactivation in fluids.

Further, high power LEDs are placed along the length of each static mixer element with each LED supplying a discrete beam of energy to the static mixer. The power level of each discrete beam is adjusted so that the total energy absorbed as each fluid element passes through each static mixer element is the sum of the individual LED beam energies and is equal to the required energy in joules/cm\hat{ }2 to activate the photochemical.

Further there has been shown two peristaltic pumps used to introduce the biological fluid and the photochemical separately into a static mixer at respective flow rates to produce the desired concentration of the photochemical after mixing to a virucidal or bacteriocidal level pre-illumination.

The use of a static mixer entrained in a column the diameter of which makes a tight fit with the wings of the mixer allows the unit to be employed to remove substances from the liquid/suspension passing through the column. Conversely, the static mixer can be employed to bring two substances into immediate contact for thorough admixture.

When used as described in the first instance, i.e.:, to remove substance(s) from material passing through a column, the adsorbent may be bound to the surfaces of the static mixer and/or the inner portion of the surrounding column. Passage of the reactant can be controlled by a pump so that the contact time between the adsorbent and substance to be removed is optimized.

For the case of using the static mixer to bring two substances together quickly for admixture, for example, latex particles may be used to which an antibody or other plasma fraction is to be bound. A pump can maintain the volume flow of each to be added to the column. When introduced, rapid mixing/contact occurs and the latex is coated/sensitized with the agent. An illustration of this can be seen in coating latex particles with an immunoglobin such as IgG resulting in a reagent for the detection of rheumatoid factor.

The static mixer/column can serve as a rapid means of washing particles free of undesired substances, for example. By repeated circulation through the system, fresh buffer or aqueous solution can be applied to rinse particles; the particles may be of chemical origin (latex) or human, animal, or bacterial/plant (red/white blood cells, erythrocytes, E. coli, yeast). After completion of washing the particulate matter can be collected by filtration or appropriate laboratory means such as centrifugation.

It is known that the chemical formulation of some polymers is such that they have an affinity to bind substances with which they come into contact. Thus, a column with a static mixer is used to bind the desired chemical while not deleteriously affecting the remainder of the circulating liquid/suspension. An example of such a polymer is polystyrene. Moreover, the constructs of the column/mixer can be modified so that their affinities for certain chemical moities are enhanced.

In a further embodiment, the static mixer arrays, photochemical/endotoxin removal filters, photochemical storage containers, final product container and interconnecting tubing, fittings and valves may be combined to create a closed, sterile disposable device.

This disposable is inserted into a programmable, illuminated processing device designed to accept the disposable, place it in intimate contact with light sources, pump fluids with peristaltic type pumps or other non-invasive pumps and non-invasive valves (ie: pinch valves).

An instrument (FIG. 7) may be employed to take measurements of, and to monitor and record, illumination intensity, fluid flows, temperatures and status.

In a further embodiment, a load cell (not shown) is provided to measure the initial weight of the sample in order to calculate and control the delivered volume of sample and photochemical.

It has been shown that the respective flows of sample and photochemical reagent are controlled so as to maintain the concentration of photochemical in the sample by means of a static mixer prior to entering the illumination zone.

LEDs of at least two different wavelengths may be used to optimize the absorption of light energy by the photochemical in the presence of the sample. Further, the reflector for the illumination devices may be a diffuse white coating of the surrounding housing. The reflector may also comprise a reflecting coating containing minute glass spheres to improve the reflection efficiency. In addition, the reflecting medium may comprise an array of retroflector panels such as used in automotive taillights and road signs.

It will be appreciated that with the use of porphyrin derivative photochemicals in common use for photoactivation purposes, at least one of the LED wavelengths is in the blue region where the absorption peak of the photochemical and the absorption peak of hemoglobin (for red blood samples) essentially coincide achieving an efficient balance of energy absorption by the photochemical and energy absorption by hemoglobin.

It will be further appreciated that while a static mixer is provided in one embodiment, an active mixer comprising a motorized mixing element can by utilized in a transparent chamber for mixing the fluids while exposing the fluids during the mixing to incident light for inactivation the pathogens. The mixing element is moved at a rate to provide the desired stretching, interchanging and folding of layers without mechanically deleteriously affecting the characteristics of the fluid. Such a mixer may for example comprise a slowly moving, e.g., rotating mixing element such as a conventional fan blade for example. The blade is shaped and sized to move all fluid against the chamber transparent wall portion. The fluid is continuously in motion so that substantially all of the fluid is exposed to the chamber transparent wall in a reasonable time period for the inactivation as determined empirically for a given implementation. Fluid may be continuously flowed into and out of this chamber. Of course, successive such chambers may also be provided each with its own corresponding light source as may desired for a given implementation.

The helical static mixers disclosed herein may, in the alternative, be coupled to a magnetic portion in the conduit, such as by bonding and so on, and rotated or axially displaced by an external armature coupled to a driving current. A rotating or an axially displaced helix operates on the fluid similarly as fluid flowing therethrough. This would eliminate the need for the pumps as the moving helical portions would flow the fluid in response to their motion. The tubes and mixers would be disposable leaving the armature in place for receiving new tube mixers. Since the mixers are rotating, their shape may vary from such helixes. With respect to axially displaced mixers, these may process fluids in the conduit in a batch process over a time period and the fluid then drained and new fluid added or the mixers disposed of. In the case of axially displaced mixers, these may be flexible so as to move along curved tubes over a desired length in which it is calculated that the pathogens have been in-activated. In addition, the rotating or axially displaced mixers may complement the pumped fluid flow to provide enhanced mixing in a small space.

It will occur that modifications may be made to the disclosed embodiments by one of ordinary skill such as shape, orientation and number of mixing elements. It is intended that the scope of the invention be defined by the appended claims.

What is claimed is:

1. A photo inactivation apparatus for a contaminated fluid containing pathogens to be inactivated comprising:
a first fluid static mixing device having at least a partially transparent wall portion for receiving a flowing first fluid comprising the contaminated fluid including a photo inactivation agent mixed therein, the device having a predetermined geometry to continuously expose substantially all of the mixed first flowing fluid therein to the transparent wall portion; and
a light source for applying incident light to the mixing device transparent wall portion and to the exposed first fluid to activate the pathogen inactivation agent throughout the fluid to thereby inactivate substantially all said pathogens in said first mixed fluid.

2. The apparatus of claim 1 wherein the predetermined geometry includes a plurality of stationary mixing elements serially arranged in at least a partially transparent conduit for continuously stretching, folding and interchanging different layers of the fluid during the exposure to the incident light so that substantially all of the fluid receives sufficient illumination from the light source during the exposure for the inactivation.

3. The apparatus of claim 1 wherein the light source comprises a plurality of light emitting diodes (LED).

4. The apparatus of claim 3 including a cooling device for cooling the LEDs by thermal regulation.

5. The apparatus of claim 4 wherein the cooling device includes a thermoelectric device.

6. The apparatus of claim 1 wherein the static mixing device comprises a static mixer comprising one of a partially transparent, totally transparent or opaque static mixer in at least one partially optically transparent conduit for receiving the flowing fluid.

7. The apparatus of claim 6 wherein the static mixer and the transparent portion of the conduit have a predetermined length and volume sufficient to inactivate substantially all of the pathogens in the fluid during the flow of the fluid through the conduit in the presence of said incident light.

8. The apparatus of claim 1 including a pump for flowing the fluid in the static mixing device at a predetermined flow rate.

9. The apparatus of claim 1 further including a second fluid static mixing device for receiving the contaminated fluid and for receiving the photo inactivation agent to produce said mixed fluid and photo inactivation agent, said second mixing device having the same geometry as the first device.

10. The apparatus of claim 1 wherein the static mixing device comprises a series of elements, each element having static mixing sections, each section comprising first and second helices in a different relative orientation.

11. The apparatus of claim 10 wherein the first and second helices are each an approximate 180° portion of a helix.

12. The apparatus of claim 1 wherein in the first mixing device comprises a plurality of transparent tube portions connected in at least one of parallel or series, each portion containing one of a transparent or opaque static fluid mixer.

13. The apparatus of claim 1 wherein the first static mixing device comprises a transparent tube portion forming the wall portion and a mixer in the tube, the mixer comprising a series of elements, wherein each element comprises first and second mirror image mixing sections.

14. The apparatus of claim 13 wherein the elements each have a given orientation, adjacent elements each having the same orientation.

15. The apparatus of claim 14 wherein all of the elements are identical and comprise a pair of helix sections in fixed relative position and rotated relative to each other about a common longitudinal axis of the tube, each section having the same orientation about the longitudinal axis.

16. The apparatus of claim 15 wherein adjacent helix sections are in reverse mirror image relation.

17. The apparatus of claim 16 wherein the sections are coaxial about the axis, adjacent sections are each rotated 90° relative to each other about the axis, alternate sections having the same orientation about the axis.

18. The apparatus of claim 15 wherein the mixer sections each comprise sheet material with a helical portion channel on opposite sides of the material.

19. The apparatus of claim 13 wherein the first and second portions are in reverse orientation and each comprise a 180° segment of a helix.

20. The apparatus of claim 19 wherein the first and second sections are rotated relative to each other about a common longitudinal axis.

21. The apparatus of claim 1 including means for heating the received first fluid

22. A static mixer device for inactivation of pathogens in a fluid flowing in a conduit comprising:
a first conduit having an optically transparent wall portion, the conduit for receiving a flowing fluid containing pathogens; and
a first static mixer in the conduit forming a first mixing device having a fluid output, the first static mixer comprising a series of first helical elements, wherein each element comprises first and second helical sections of different orientations for exposing substantially all of the flowing fluid to incident light applied to said wall portion for said inactivation.

23. The static mixer of claim 22 wherein the conduit defines a longitudinal axis, the elements each having a given angular orientation relative to the axis, adjacent elements having the same angular orientation.

24. The static mixer of claim 23 wherein all of the elements are identical and each element comprises a pair of helix sections rotated relative to each other about the longitudinal axis.

25. The static mixer of claim 24 wherein adjacent helix portions are in reverse mirror image relation.

26. The static mixer of claim 24 wherein the sections are coaxial about the axis, adjacent sections are each rotated 90° relative to each other about the axis, alternate sections having the same angular orientation about the axis.

27. The static mixer of claim 22 wherein the mixer sections each comprise sheet material with a helical section channel on opposite sides of the material.

28. The static mixer of claim 22 wherein the first and second helical sections are in reverse orientation and each section comprises a 180° segment of a helix.

29. The static mixer of claim 22 wherein the first and second helical sections are rotated 90° relative to each other about a common longitudinal axis.

30. The static mixer of claim 22 wherein the first and second sections have a given relative angular orientation to each other about a common axis defined by the conduit, alternate sections of adjacent sections having the same angular orientation relative to said axis.

31. The static mixer of claim 22 including a pump for pumping the flowing fluid into the conduit.

32. The static mixer of claim 22 comprising:
a second conduit fluid output coupled to an input of the first conduit and a second static mixer in the second conduit forming a second mixing device;
the second mixer comprising a series of second helical sections;
a first input to the second conduit for introducing a first fluid containing at least one pathogen;
a second input to the second conduit for introducing a photo pathogen inactivation first fluid having a given absorbence for inactivation of at least one pathogen in the first fluid in response to incident light applied to the first mixer; and
a first pump for flowing the pathogen containing first fluid into the first input and a second pump for flowing the photo inactivation second fluid into the second input.

33. The static mixer of claim 32 including a control for operating the first and second pumps at predetermined relative flow rates.

34. The static mixer of claim 32 including a plurality of LEDs optically coupled to the first static mixer for activating the photo inactivation fluid.

35. The static mixer of claim 34 wherein the LEDs have at least one wavelength and intensity.

36. The static mixer of claim 32 including a plurality of arrays of first conduits and static mixers fluidly connected in one of/or both of series and parallel.

37. The static mixer of claim 32 including a control for varying the flow rate of the pathogen containing first fluid and photo responsive second fluid, light intensity and array format of the first and second static mixers according to time constraint, chemical activation requirements and total volume of the fluid to be processed.

38. The static mixer of claim 34 including a plurality of first static mixers and mating corresponding conduit pairs, each latter pair forming a conduit section of a given length, a plurality of light emitting arrays, each array for illuminating a length of a different first static mixer section.

39. The static mixer of claim 34 including a control for selecting the radiant power levels of the LEDs to match the absorbance characteristics of the pathogen inactivation fluid.

40. The static mixer of claim 34 wherein the first static mixer comprises a plurality of static mixers and mating conduit pairs, each pair having a given length, the LEDs being located along the given length with each LED applying a discrete beam of radiant energy to the corresponding static mixer pair.

41. The static mixer of claim 40 wherein the first fluid and each static mixer comprises a plurality of respective elements, the power level of each discrete beam has a value wherein the total energy absorbed as each fluid element passes through each static mixer element is the sum of the individual LED beam energies and is equal to the required energy in joules/cm² to activate the photo inactivation fluid.

42. The static mixer of claim 32 wherein the first and second pumps are each a peristaltic pump for flowing the respective fluids at a respective flow rate to produce a predetermined concentration of the photo inactivation fluid after mixing to a virucidal or bacteriocidal level pre-illumination.

43. The static mixer of claim 32 wherein the fluid output of the first static mixer device includes photochemicals and endotoxins, further including a resin column coupled to receive the fluid output for removal of the photochemical and endotoxins resident in the fluid output.

44. The static mixer of claim 43 wherein the resin column comprises a static mixer packed with the resin.

45. The static mixer of claim 43 wherein the fluid output of the first static mixer device includes photochemicals and endotoxins, further including a further mixer device coupled to the fluid output for removal of the photochemical and endotoxins resident in the fluid output, the further mixer device comprising a coated static mixer in a conduit wherein the coating comprises a resin attractant material for the photochemical and means of endotoxin removal.

46. The static mixer of claim 22 wherein at least a portion of the conduit and a portion of the mixer are optically transparent.

47. A static mixer device comprising:
a first conduit having at least an optically transparent wall portion for receiving a flowing fluid containing pathogens; and
a first static mixer in the conduit forming a first mixing device having a fluid output, the first static mixer comprising a series of first helical elements, wherein each element comprises first and second helical sections of different orientations for exposing the flowing fluid substantially uniformly to incident light applied to said wall portion for said inactivation.

48. The static mixer device of claim 47 wherein the first static mixer has a transparent portion.

49. The static mixer device of claim 47 wherein the conduit is a serpentine tube having an array of parallel transparent portions, each parallel portion including a static mixer therein.

50. The static mixer device of claim 49 wherein the static mixers in each portion are transparent.

51. A method of inactivation of pathogens in a fluid comprising flowing the fluid containing a photo inactivation agent through a static mixing device including a housing that has at least a partially transparent wall portion containing a static mixer while exposing the transparent wall portion to incident light for activating the agent to inactivate substantially all of the pathogens in the fluid.

52. A method of filtering a body fluid containing inactivated pathogens comprising filtering the fluid with a static mixing device containing filtering materials.

53. The method of claim 52 including providing a membrane in the device coated with the filtering materials.

54. The method of claim 52 including filling the device with a filtering resin forming the filtering materials.

55. The method of claim 52 wherein the mixing device includes a static mixer, the method including coating the static mixer with filtering materials.

56. The method of claim 55 wherein the filtering materials are adsorption resin particles.

57. The method of claim 52 wherein the mixing device includes a housing having a chamber in which the fluid flows, the method including coating the housing in the chamber with the filtering materials.

58. The method of claim 57 wherein the filtering materials include adsorption resin particles.

59. A filtering device for filtering a flowing body fluid containing inactivated pathogens and an inactivation agent comprising:
a static mixing device for receiving the flowing fluid; and
filtering materials in the device for filtering the inactivated pathogens and agent.

60. The device of claim 59 wherein the filtering materials comprise resin particles.

61. The device of claim 60 wherein the mixing device has a chamber defined by a wall and including a static mixer therein, and wherein particles are coated on the static mixer and on the chamber wall.

62. The device of claim 60 wherein the mixing device has a chamber including a static mixer in the chamber and wherein the particles are packed inside of the chamber.

63. In combination:
a static mixing device having at least a transparent portion for receiving a flowing body fluid containing pathogens and a pathogen photo inactivation agent for receiving incident light to inactivate the pathogens; and
a filter coupled to the device and containing a static mixer and filter material for filtering the agent and inactivated pathogens from the fluid.

64. The device of claim 63 wherein the device comprises a housing having a chamber and a static mixer element in the chamber, at least the housing having a transparent portion.

65. The device of claim 64 wherein the static mixer has at least a transparent portion adjacent to the housing transparent portion.

66. A method for inactivating pathogens in a fluid containing a photo inactivating agent comprising:
the step of providing a fluid mixer in a chamber having an optically transparent wall portion and for mixing the fluid containing the pathogens, the mixer for stretching, folding and interchanging layers of the fluid so that substantially all of the fluid is exposed to the chamber wall portion over a period of time; and
exposing the chamber wall portion to sufficient light intensity at at least one wavelength during the period to substantially expose all of said layers to the light and thus to substantially expose all said agent to the light during the stretching, folding and interchanging to substantially inactivate all of the pathogens in the fluid.

67. The method of claim 66 wherein the providing the mixer step includes mixing the fluid with a static mixing element.

68. The method of claim 66 wherein the providing the mixer step includes actively mixing the fluid with a moving mixer element.
